(19) United States
(12) Patent Application Publication
(10) Pub. No.: US 2007/0207971 A1
(43) Pub. Date: Sep. 6, 2007

(54) Neuraminidase inhibitor

(76) Inventors: Lee-Chiang Lo, Taipei (TW); Shih-Hsiung Wu, Taipei (TW)

Correspondence Address: OCCHIUTI ROHLICEK & TSAO, LLP, 10 FAWCETT STREET, CAMBRIDGE, MA, 02138, US

(21) Appl. No.: 11/368,589

(22) Filed: Mar. 6, 2006

(51) Int. Cl. A61K 31/7052

(52) U.S. Cl. 514/25; 536/53

(57) ABSTRACT

This invention relates to a method of treating an infection with an influenza virus. The method includes administering to a subject in need thereof an effective amount of a compound of formula (I): 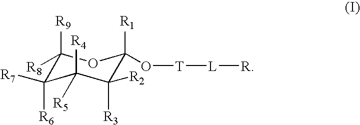 Each variable in this formula is defined in the specification.

### BACKGROUND

Influenza viruses, which cause upper respiratory tract infections in human, have long been a major threat to public health. It is estimated that 10-20% of the general population are infected with influenza viruses each year.

Influenza viruses are typically spherical particles having a diameter of about 30-120 nm. Neuraminidase (also known as N-acylneuraminosyl glycohydrolase) is a surface antigen involved in the budding process during the propagation of influenza viruses. It hydrolyzes the glucosidic linkage of sialic acid in glycoconjugates. Although influenza viruses frequently mutate to avert attacks from the immune system of the hosts, the catalytic activity of neuraminidase has to be maintained for successful propagation. This feature makes neuraminidase an excellent target for inhibiting influenza virus growth.

X-ray crystallographic information about the active site of neuraminidase has revealed important residues involved in the recognition and binding of sialic acid. See, e.g., Varghese et al., Proteins Struct. Funct. Genet. 1992, 14, 327. This has assisted in the development of several reversible neuraminidase inhibitors such as zanamivir, which was already approved for treating influenza viral infection. Note that zanamivir binds to neuraminidase via a non-covalent interaction. There remains a need to develop an neuraminidase inhibitor that binds to neuraminidase via a covalent bonding to minimize the effect of virus mutation on its inhibitory activity.

### SUMMARY

This invention is based on the discovery that certain neuraminidase inhibitors can be used to detect an influenza virus and inhibit influenza virus growth.

In one aspect, this invention features a compound of formula (I): 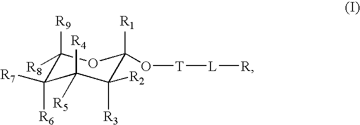
In this formula, T is arylene or C₇-C₂₀ arylalkylene; L is -L₁-L₂-L₃-; L₁ being deleted, —C(O)N(Rₐ₁)—, or —N(Rₐ₁)C(O)—; L₂ being deleted or C₁-C₃₀ alkyl optionally containing 1-10 heteroatoms, —C(O)N(Rₐ₂)—, or —N(Rₐ₂)C(O)—; and L₃ being deleted or —N(Rₐ₃)—; R is —C(O)—R_{b1}, —S(O)₂—R_{b1}, —N(R_{b1})(R_{b2}), —N₃, C₂-C₁₀ alkynyl, or heteroaryl; R₁ is COOR_{c1}; each of R₂ and R₃, independently, is H, OR_{d1}, or C₁-C₁₀ alkyl; one of R₄ and R₅ is ORₑ₁, and the other of R₄ and R₅ is H, ORₑ₂, or C₁-C₁₀ alkyl; one of R₆ and R₇ is N(R_{f1}R_{f2}), and the other of R₆ and R₇ is H, OR_{f3}, or C₁-C₁₀ alkyl; and one of R₈ and R₉ is C₁-C₁₀ alkyl substituted with OR_{g1}, and the other of R₈ and R₉ is H, OR_{g2}, or C₁-C₁₀ alkyl; in which each of Rₐ₁, Rₐ₂, Rₐ₃, R_{b1}, R_{b2}, R_{c1}, R_{d1}, Rₑ₁, Rₑ₂, R_{f1}, R_{f2}, R_{f3}, R_{g1}, and R_{g2}, independently, is H, C₁-C₁₀ alkyl, C₃-C₂₀ cycloalkyl, C₃-C₂₀ heterocycloalkyl, heteroaryl, aryl, or —C(O)R′; R′ being H or C₁-C₁₀ alkyl; or a salt thereof.

Referring to formula (I), a subset of the compounds described above are those in which T is arylene. In these compounds, T can be phenylene substituted with CHF₂, L₁ can be —N(Rₐ₁)C(O)—, L₂ can be C₁-C₃₀ alkyl containing —C(O)N(Rₐ₂)— and —N(Rₐ₂)C(O)—, L₃ can be —N(Ra₃)—, R can be —C(O)—R_{b1}, R_{b1} can be C₁-C₁₀ alkyl substituted with heteroaryl, R₁ can be COOH, R₂ can be H, R₃ can be H, R₄ can be H, R₅ can be OH, R₆ can be H, R₇ can be NHAc, R₈ can be C₁-C₁₀ alkyl substituted with three OH, and R₉ can be H. Examples include: 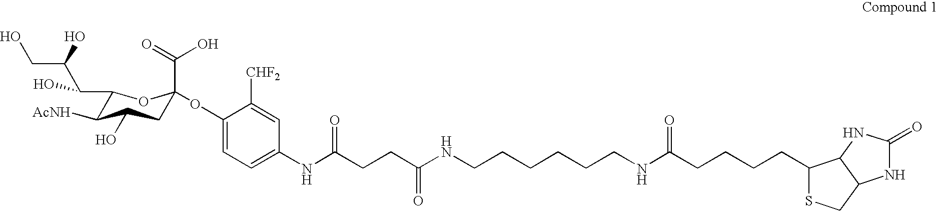
and a salt thereof.

Referring to formula (I), another subset of the compounds described above are those in which T is C₇-C₂₀ arylalkylene. In these compounds, T can be 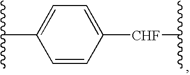
L₁ can be —C(O)N(Rₐ₁)—, L₂ can be C₁-C₃₀ alkyl containing 1-10 heteroatoms, L₃ can be —N(Rₐ₃)—, R can be —C(O)—R_{b1}, and R_{b1} can be C₁-C₁₀ alkyl substituted with heteroaryl.

The term “alkyl” refers to a saturated or unsaturated, straight or branched hydrocarbon moiety, such as —CH₃, —CH₂—CH═CH₂, or branched —C₃H₇. The term “alkynyl” refers to a straight or branched hydrocarbon moiety having a triple bond, such as ethynyl. The term “cycloalkyl” refers to a saturated or unsaturated, non-aromatic, cyclic hydrocarbon moiety, such as cyclohexyl or cyclohexen-3-yl. The term “heterocycloalkyl” refers to a saturated or unsaturated, non-aromatic, cyclic moiety having at least one ring heteroatom (e.g., N, O, or S), such as 4-tetrahydropyranyl or 4-pyranyl. The term “aryl” refers to a hydrocarbon moiety having one or more aromatic rings. Examples of aryl moieties include phenyl (Ph), naphthyl, pyrenyl, anthryl, and phenanthryl. The term “arylene” refers to a divalent hydrocarbon moiety having one or more aromatic rings, such as phenylene. The term “arylalkylene” refers to a divalent hydrocarbon moiety containing at least one aryl group and at least one alkyl group, in which one radical is located on the aryl group and the other radical is located on the alkyl group. An example of an arylalkylene group is 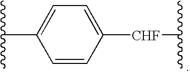
The term “heteroaryl” refers to a moiety having one or more aromatic rings that contain at least one ring heteroatom (e.g., N, O, or S). Examples of heteroaryl moieties include furyl, fluorenyl, pyrrolyl, thienyl, oxazolyl, imidazolyl, thiazolyl, pyridyl, pyrimidinyl, quinazolinyl, quinolyl, isoquinolyl and indolyl.

Alkyl, alkynyl, cycloalkyl, heterocycloalkyl, aryl, arylene, arylalkylene, and heteroaryl mentioned herein include both substituted and unsubstituted moieties, unless specified otherwise. Possible substituents on cycloalkyl, heterocycloalkyl, aryl, arylene, arylalkylene, and heteroaryl include, but are not limited to, C₁-C₁₀ alkyl, C₂-C₁₀ alkenyl, C₂-C₁₀ alkynyl, C₃-C₈ cycloalkyl, C₅-C₈ cycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, hydroxyl, halogen, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, acyl, thioacyl, acyloxy, carboxyl, and carboxylic ester. On the other hand, possible substituents on alkyl and alkynyl include all of the above-recited substituents except C₁-C₁₀ alkyl, C₂-C₁₀ alkenyl, and C₂-C₁₀ alkynyl. Cycloalkyl, heterocycloalkyl, aryl, arylene, arylalkylene, and heteroaryl can also be fused with each other.

In another aspect, this invention features a method of treating an infection with an influenza virus. The method includes administering to a subject in need thereof an effective amount of a compound of formula (I). The term “treating” or “treatment” refers to administering one or more compounds described above to a subject, who has an infection with an influenza virus, a symptom of such an infection, or a predisposition toward such an infection, with the purpose to confer a therapeutic effect, e.g., to cure, relieve, alter, affect, ameliorate, or prevent the infection with an influenza virus, the symptom of it, or the predisposition toward it.

In still another aspect, this invention features a method of detecting presence of an influenza virus in a sample. The method includes (1) contacting a sample with a compound of formula (I) and (2) determining presence of binding between the compound of formula (I) and an influenza virus, the binding being an indication of presence of the influenza virus. The method can further include attaching the compound of formula (I) to a substrate (e.g., a microtiter plate) before the contacting step. Further, the just-described detection method can include a western blot analysis or an enzyme-linked immunosorbent assay.

The compounds described above include the compounds of formula (I), as well as their salts, prodrugs, and solvates, if applicable. A salt, for example, can be formed between an anion and a positively charged group (e.g., amino) on a compound of formula (I). Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a compound of formula (I). Suitable cations include sodium ion, potassium ion, magnesium ion, calcium ion, and an ammonium cation such as tetramethylammonium ion. The compounds described above also include those salts containing quaternary nitrogen atoms. Examples of prodrugs include esters and other pharmaceutically acceptable derivatives, which, upon administration to a subject, are capable of providing active compounds. A solvate refers to a complex formed between an active compound and a pharmaceutically acceptable solvent. Examples of pharmaceutically acceptable solvents include water, ethanol, isopropanol, ethyl acetate, acetic acid, and ethanolamine.

Also within the scope of this invention is a composition containing one or more of the compounds described above for use in treating an infection with an influenza virus, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

This invention relates to certain neuraminidase inhibitors, as well as their uses for detecting an influenza virus and treating infection with an influenza virus.

In general, each of the compounds described above can contain four different groups: a recognition head, a trapping group, a linker, and a reporter group. For example, in compound 1, the recognition head is the sialic acid moiety, the trapping group is the difluoromethylphenylene group, the linker is —NHC(O)(CH₂)₂C(O)NH(CH₂)₆NH—, and the reporter group is the biotin moiety.

Take compound 1 for example. When this compound is used to inhibit the growth of an influenza virus, the recognition head directs compound 1 to the active site of neuraminidase. The enzyme then cleaves the recognition head from compound 1 by breaking the glycosidic bond to afford an intermediate containing a reactive trapping group. The reactive trapping group subsequently reacts with a nucleophile at or near the active site and thereby block it. As a result, the growth of the influenza virus is inhibited due to loss of neuraminidase activity. The above-mentioned intermediate is described in Liu et al., Angew. Chem. Int. Ed., 2005, 44:6888-6892. Another intermediate that can be generated by other compounds of the invention is described in Tsai et al., Organic Letters, 2002, 4(21):3607-3610.

Compound 1 can also be used to detect an influenza virus, such as by a Western blot analysis or an enzyme-linked immunosorbent assay. The recognition head and the trapping group are used to covalently bind the virus to compound 1 in the same manner as described above. In a Western blot analysis, the reporter group (i.e., the biotin moiety) is used to visualize (e.g., by streptavidin-conjugated peroxidase chemiluminescence) the presence of virus particles that contain neurimindase. In an enzyme-linked immunosorbent assay, the reporter group functions as a means to immobilize virus particles to a microtiter plate, e.g., through a biotin-avidin interaction. For example, virus particles are captured by covalently binding to compound 1 that is already attached to the microtiter plate through the interaction between the reporter group (i.e., the biotin moiety) and its coupling partner (e.g., containing an avidin moiety) coated on the microtiter plate. The presence of the virus can then be detected using an antibody specific to a viral antigen. Note that, when a compound of the invention is used as a drug for treating influenza viral infection, its reporter group can be simply an end-capping group (e.g., acetyl) that is not able to detect or immobilize virus particles. The linker on compound 1 reduces the steric hindrance between captured virus particles by increasing the distance between a captured virus particle and the microtiter plate, thereby increasing the amount of the captured virus particles.

Covalently binding virus particles to compound 1 allows for subsequent rigorous manipulation that may not be feasible when the virus particles are non-covalently bound to a compound.

The compounds described above can be prepared by methods well known in the art, such as those described herein and those described in Tsai et al., Organic Letters, 2002, 4(21):3607-3610. Schemes I and II shown below depicts a typical synthetic route for synthesizing certain exemplary compounds of the invention. In these two schemes, R₁-R₉ and R_{b1} are defined in the Summary section above.

As shown in Scheme I, a glucose derivative (i.e., containing a recognition head) can first be modified by replacing the hydroxyl group at C-1 position with a halogen group (e.g., chloride or bromide). The compound thus obtained can then react with 2-hydroxy-5-nitrobenzaldehyde to give intermediate A, which can be subsequently modified to form intermediate B having a amino group and a difluoromethyl group on the phenyl ring (i.e., an intermediate containing a trapping group). Intermediate B can then react sequentially with succinic anhydride and an amino compound containing a reporting group (e.g., a biotin moiety) to obtain certain compounds of this invention. Example 1 below provides a detailed description of how compound 1 was prepared based on the methods described in Scheme I.

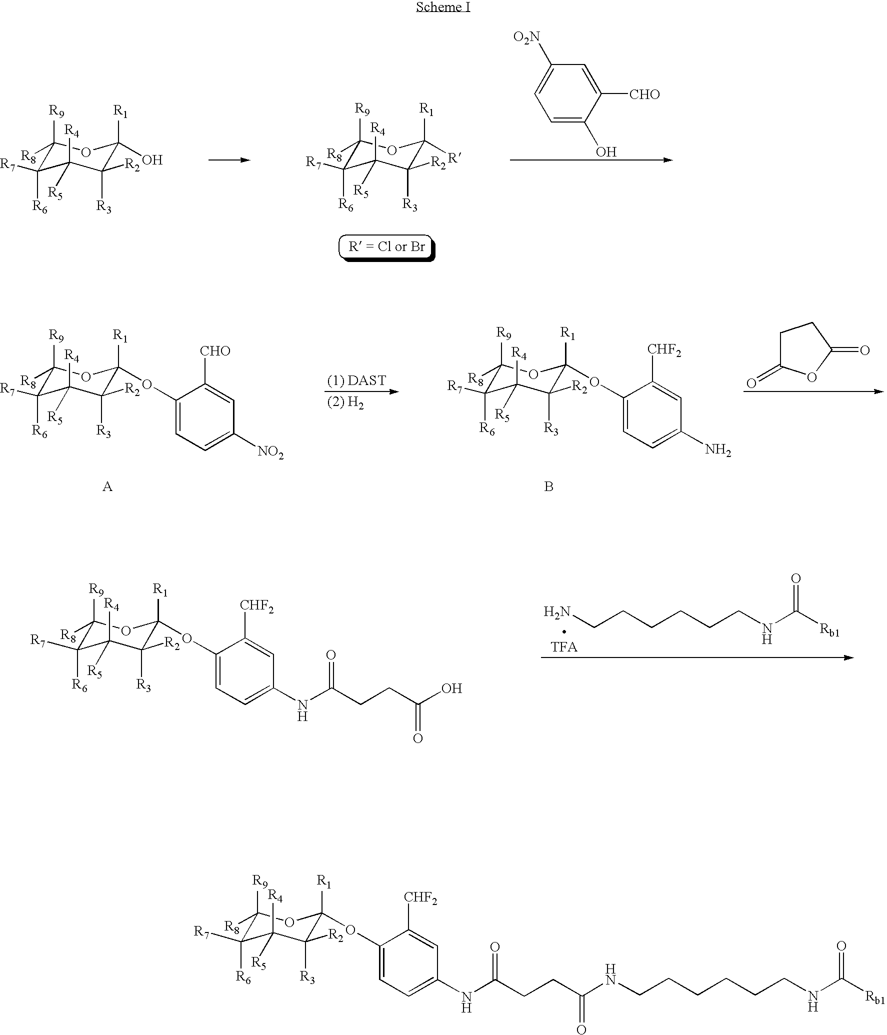

As shown in Scheme II, a glucose derivative (i.e., containing a recognition head) can first be modified by replacing the hydroxyl group at C-1 position with a halogen group (e.g., chloride or bromide) and then react with benzyl 2-(4-hydroxyphenyl)acetate to give intermediate A. Intermediate A can be subsequently modified to form intermediate B having a carboxylfluoromethyl group on the phenyl ring (i.e., containing a trapping group). Intermediate B can then react with an amino compound containing a reporting group (e.g., a biotin moiety) to obtain certain other compounds of this invention.

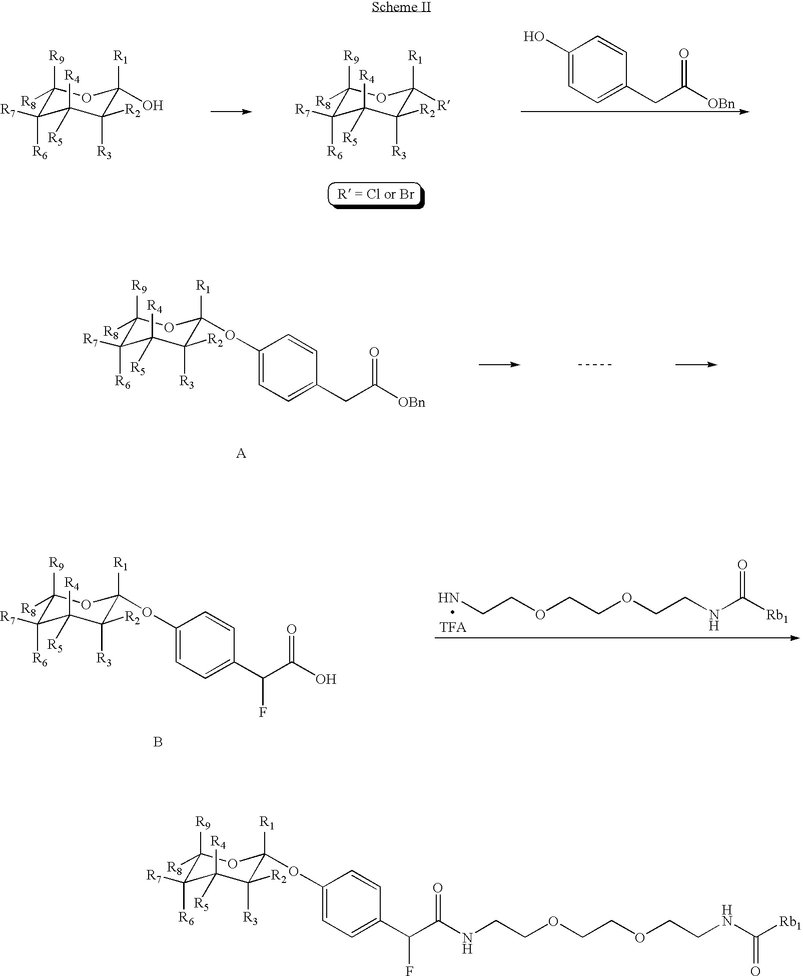

Compounds synthesized by the methods described above can be purified by methods well known in the art, e.g., column chromatography, high-pressure liquid chromatography, or recrystallization.

Other compounds described above can be prepared using other suitable starting materials through the above synthetic routes and others known in the art. The methods described above may also additionally include steps, either before or after the steps described specifically herein, to add or remove suitable protecting groups in order to ultimately allow synthesis of the compounds described above. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The compounds mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition containing an effective amount of at least one compound described above and a pharmaceutical acceptable carrier. Further, this invention covers a method of administering an effective amount of one or more of the compounds described above to a patient having an infection with an influenza virus. “An effective amount” refers to the amount of an active compound that is required to confer a therapeutic effect on the treated subject. Effective doses will vary, as recognized by those skilled in the art, depending on the types of diseases treated, route of administration, excipient usage, and the possibility of co-usage with other therapeutic treatment.

This invention also covers a method of detecting presence of an influenza virus in a sample by (1) contacting a sample with a compound described above, and (2) determining presence of binding between the compound and an influenza virus, the binding being an indication of presence of the influenza virus. For example, the method can be a western blot analysis or an enzyme-linked immunosorbent assay.

To practice the treatment method of the present invention, a composition having one or more compounds described above can be administered parenterally, orally, nasally, rectally, topically, or buccally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active compounds described above can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound described above. Examples of other carriers include colloidal silicon oxide, magnesium stearate, cellulose, sodium lauryl sulfate, and D&C Yellow #10.

The compounds described above can be preliminarily screened for their efficacy in treating an infection with an influenza virus by an in vitro assay (See Examples 2-4 below) and then confirmed by animal experiments and clinical trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of Compound 1

Compound 1 was synthesized in the manner shown in Scheme III below:

All reagents and starting materials were obtained from commercial suppliers (Acros, Morris Plains, N.J.; Aldrich, St. Louis, Mo.; and Merck, Whitehouse Station, N.J.) and were used without further purification. IR spectra were recorded on a Nicolet 550 series II spectrometer. ¹H, ¹⁹F, and ¹³C NMR were recorded using a Brucker AC-300 or Bruker Avance 400 spectrometer. The proton and carbon chemical shifts are given in ppm using CDCl₃ (δ_{H} at 7.24 ppm and δ_{C} at 77.0 ppm) as internal standard. High resolution mass spectra were recorded using a JEOL-102A mass spectrometer. Analytical TLC (silica gel, 60F-54, Merck, Whitehouse Station, N.J.) and spots were visualized under UV light and/or using phosphomolybdic acid-ethanol. Column chromatography was performed using Kiesegel 60 (70-230 mesh) silica gel (Merck, Whitehouse Station, N.J.).

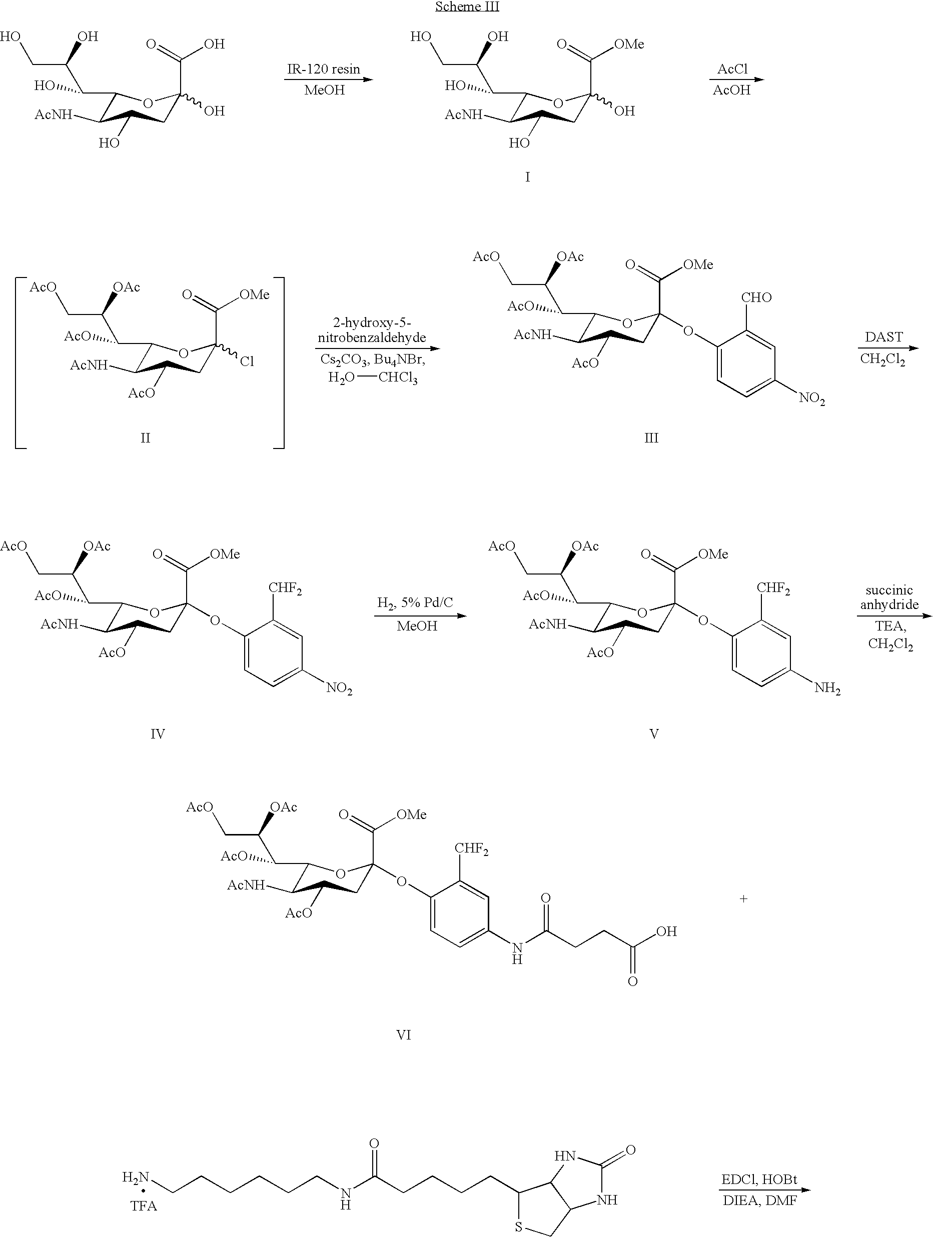

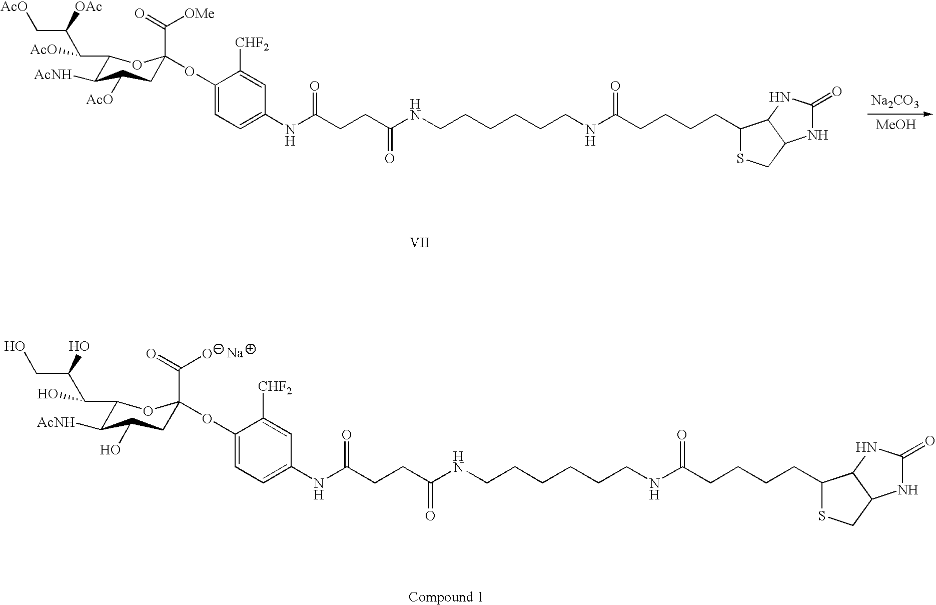

N-Acetylneura-minic acid (1.00 g, 3.2 mmol) was suspended in 25 mL of anhydrous MeOH. Amberlite IR-120 (H+) resin (0.67 g) was added to the above mixture. The reaction mixture was then stirred until the suspension became a clear solution. After removal of the resin by filtration, the filtrate was concentrated under reduced pressure. Ether was added to the solution thus obtained to form a white solid. The solid was subsequently collected by filtration to afford Intermediate I: methyl 5-acetamido-3,5-dideoxy-D-galacto-2-nonulopyranosonate (0.96 g, 92%).

22 mL of freshly distilled acetyl chloride was added slowly to an ice-cooled solution of Intermediate I (0.96 g, 3.0 mmol) in acetic acid (12 mL). The reaction mixture was stirred for 48 hours. It was then concentrated by removing substantially all volatiles to give Intermediate II, which was used for the next step without further purification.

A solution of 2-hydroxy-5-nitrobenzaldehyde (1.50 g, 9.0 mmol) in 150 mL of Cs₂CO₃ (0.1 M) was added to a solution of Intermediate II (3.0 mmol) and tetrabutylammonium bromide (2.10 g, 6.6 mmol) in 100 mL of CHCl₃. The biphasic reaction mixture was stirred at room temperature overnight. When no more starting materials were observed (e.g., after about 12 hours), the organic layer was separated and removed. The aqueous layer was extracted three times with CHCl₃. The CHCl₃ extracts were combined, washed with a saturated NaCl solution, and dried over anhydrous Na₂SO₄ to give a crude product. The crude product was purified by silica gel column chromatography using hexane/EtOAc (6/4) as an eluent to afford Intermediate III: methyl (2-O-(2-formyl-4-nitro)phenyl-5-acetamido-4,7,8,9-tetra-O-acetyl-3,5-dideoxy-D-glycero-α-D-galacto-2-nonulopyranosid)onate (1.20 g, 67% from Intermediate I).

Melting point: 192-194° C.; IR (KBr): 3257, 1759, 1739, 1646 cm⁻¹; ¹H-NMR (CDCl₃, 300 MHz): δ 10.36 (s, 1 H, CHO), 8.62 (d, J=2.9 Hz, 1 H, aromatic), 8.36 (dd, J=9.1, 2.9 Hz, 1 H, aromatic), 7.39 (d, J=9.1 Hz, 1 H, aromatic), 5.57 (d, J=10.0 Hz, 1 H, NH), 5.34-5.29 (m, 2 H, H-7+H-8), 4.99 (ddd, J=11.7, 10.4, 4.7 Hz, 1 H, H-4), 4.61 (d, J=10.9 Hz, 1 H, H-9), 4.19-3.99 (m, 3 H, H-5+H-6+H-9′), 3.63 (s, 3 H, OCH₃), 2.79 (dd, J=12.2, 4.7 Hz, 1 H, H-3e), 2.33 (dd, J=12.2, 11.7 Hz, 1 H, H-3a), 2.13 (s, 3 H, OAc), 2.06 (s, 3 H, OAc), 2.02 (s, 3 H, OAc), 2.00 (s, 3 H, OAc), 1.88 (s, 3 H, NAc); ¹³C-NMR (CDCl₃, 100 MHz): δ187.0 (CH), 170.8 (C), 170.5 (C), 170.3 (C), 170.1 (C), 170.0 (C), 167.6 (C), 160.0 (C), 143.6 (C), 130.5 (CH), 126.3 (C), 124.2 (CH), 119.4 (CH), 100.0 (C), 73.9 (CH), 68.0 (CH), 67.8 (CH), 66.9 (CH), 62.2 (CH₂), 53.6 (CH₃), 49.4 (CH), 38.6 (CH₂), 23.2 (CH₃), 21.0 (CH₃), 20.8 (CH₃), 20.7 (CH₃); MS m/z (%): 641 (7, M⁺+H), 474 (18), 414 (100); HRMS calcd for C₂₇H₃₃N₂O₁₆: 641.1830, found 641.1828.

Diethylaminosulfur trifluoride (DAST, 1.6 mL, 10.0 mmol) was slowly added through a syringe to an ice-cooled solution of Intermediate III (1.60 g, 2.5 mmol) in 6 mL of anhydrous CH₂Cl₂. The reaction mixture was stirred overnight. When no more starting materials were observed, the mixture was cooled and quenched by adding MeOH. The mixture thus obtained was concentrated to give a crude product, which was subsequently purified by silica gel column chromatography to afford Intermediate IV: methyl (2-O-(2-difluoromethyl-4-nitro)phenyl-5-acetamido-4,7,8,9-tetra-O-acetyl-3,5-di-deoxy-D-glycero-α-D-galacto-2-nonulopyranosid)onate (778.4 mg, 47%). Melting point: 72-76° C.; IR (KBr): 3456, 1739, 1660 cm⁻¹; ¹H-NMR (CDCl₃, 300 MHz): δ 8.42 (d, J=2.7 Hz, 1 H, aromatic), 8.29 (dd, J=9.3, 2.7 Hz, 1 H, aromatic), 7.37 (d, J=9.3 Hz, 1 H, aromatic), 6.86 (t, J=54.9 Hz, 1 H, CHF₂), 5.44 (d, J=9.9 Hz, 1 H, NH), 5.34-5.29 (m, 2 H, H-7+H-8), 4.97 (ddd, J=11.5, 10.5, 4.6 Hz, 1 H, H-4), 4.60 (d, J=11.2 Hz, 1 H, H-9), 4.21-4.02 (m, 3 H, H-5+H-6+H-9′), 3.62 (s, 3 H, OCH₃), 2.75 (dd, J=12.9, 4.6 Hz, 1 H, H-3e), 2.30 (dd, J=12.9, 11.5 Hz, 1 H, H-3a), 2.15 (s, 3 H, OAc), 2.07 (s, 3 H, OAc), 2.02 (s, 3 H, OAc), 2.01 (s, 3 H, OAc), 1.90 (s, 3 H, NAc); ¹³C-NMR (CDCl₃, 100 MHz): δ 170.6 (C), 170.5 (C), 170.4 (C), 170.0 (C), 169.8 (C), 167.4 (C), 156.4 (C), 143.1 (C), 127.7 (CH), 125.2 (C), 122.4 (CH), 118.3 (CH), 109.9 (CH, t, J=237.2 Hz), 100.0 (CH), 73.7 (CH), 68.0 (CH), 67.9 (CH), 66.8 (CH), 62.1 (CH2), 53.4 (CH₃), 50.6 (CH), 38.4 (CH₂), 23.0 (CH₃), 21.2 (CH₃), 20.8 (CH₃), 20.6 (CH₃), 20.5 (CH₃); MS m/z (%): 663 (67, M⁺+H), 603 (25), 414 (100); HRMS calcd for C₂₇H₃₃F₂N₂O₁₅: 663.1849, found 663.1808.

Pd/C (5%, 5 mg) was added to a solution of Intermediate IV (113.7 mg, 0.18 mmol) in 5 mL of MeOH. The reaction system was flushed with H₂ three times. The reaction mixture was kept under H₂ atmosphere with a balloon and stirred overnight. The Pd/C catalyst was then removed by filtration through Celite 535. The filtrate was concentrated to afford Intermediate V: methyl (2-O-(2-difluoromethyl-4-amino)phenyl-5-acetamido-4,7,8,9-tetra-O-acetyl-3,5-dideoxy-D-glycero-α-D-galacto-2-nonulopyranosid)onate (108.2 mg, 95%). Melting point: 82-86° C.; ¹H-NMR (CDCl₃, 300 MHz): δ 7.08 (d, J=8.8 Hz, 1 H, aromatic), 7.01-6.64 (m, 3 H, aromatic+CHF₂), 5.34 (s, 2 H, H-7+H-8), 5.19 (d, J=10.0 Hz, 1 H, NH), 4.92 (ddd, J=12.5, 10.4, 4.6 Hz, 1 H, H-4), 4.33-4.02 (m, 4 H, H-5+H-6+H-9), 3.62 (s, 3 H, OAc), 2.68 (dd, J=12.5, 4.6 Hz, 1 H, H-3e), 2.19-2.12 (m, 4 H, H-3a+OAc), 2.11 (s, 3 H, OAc), 2.03 (s, 3 H, OAc), 2.01 (s, 3 H, OAc), 1.89 (s, 3 H, NAc); ¹³C-NMR (CDCl₃, 100 MHz): δ 170.9 (C), 170.6 (C), 170.2 (C), 170.0 (C), 167.4 (C), 143.4 (C), 122.1 (CH), 118.1 (CH), 111.9 (CH), 111.3 (CH, t, J=234.1 Hz), 100.9 (C), 73.2 (CH), 68.9 (CH), 68.7 (CH), 67.2 (CH), 62.0 (CH₂), 53.0 (CH₃), 49.5 (CH), 37.7 (CH₂), 23.3 (CH₃), 21.0 (CH₃), 20.8 (CH₃), 20.8 (CH₃), 20.7 (CH₃); MS m/z (%): 633 (24, M⁺+H), 414 (100); HRMS calcd for C₂₇H₃₅F₂N₂O₁₃: 633.2107, found 633.2134.

Triethyl amine (TEA, 0.10 mL, 0.71 mmol) was added to a solution of Intermediate V (237 mg, 0.37 mmol) and succinic anhydride (50 mg, 0.50 mmol) in CH₂Cl₂ (5.0 mL). The mixture was stirred at room temperature for 3 hours, diluted with EtOAc (150 mL), and washed successively with 5% aqueous citric acid (10 mL×3) and water (10 mL×2). The aqueous layers were combined and extracted once with EtOAc (150 mL). The EtOAc layer was washed again with water (10 mL×2). The organic layer was dried over anhydrous Na₂SO₄, filtered, and concentrated under reduced pressure to afford Intermediate VI: 3-acetoxy-5-acetylamino-2-[4-(3-carboxy-propionylamino)-2-difluoromethyl-phenoxy]-6-(1,2,3-triacetoxy-propyl)-tetrahydro-pyran-2-carboxylic acid methyl ester (258 mg, 94%) as a light brown foam. ¹H-NMR (CD₃OD, 400 MHz): δ 7.84 (d, J=2.2 Hz, 1 H, aromatic), 7.56 (dd, J=9.0, 2.2 Hz, 1 H, aromatic), 7.26 (d, J=9.0 Hz, 1 H, aromatic), 6.96 (t, J=55.3 Hz, 1 H, CHF₂), 5.38-5.36 (m, 2 H, H-7+H-8), 4.91-4.89 (m, 1 H), 4.49 (d, J=10.9 Hz, 1 H), 4.29 (d, J=11.3 Hz, 1 H), 4.10 (dd, J=12.3, 2.0 Hz, 1 H), 4.03 (dd, J=10.5, 10.5 Hz, 1 H), 3.64 (s, 3 H, OCH₃), 2.80 (dd, J=13.0, 4.7 Hz, 1 H, H-3e), 2.66 (s, 4 H), 2.16-2.15 (m, 4 H, H-3a+OAc), 2.10 (s, 3 H, OAc), 2.01 (s, 3 H, OAc), 1.99 (s, 3 H, OAc), 1.86 (s, 3 H, NAc); ¹³C-NMR (CD₃OD, 100 MHz): δ 176.6 (C), 173.9 (C), 173.1 (C), 172.7 (C), 172.0 (C), 171.9 (C), 171.7 (C), 169.2 (C), 149.2 (C), 136.9 (C), 127.6 (C), 124.5 (CH), 121.9 (CH), 118.8 (CH), 112.9 (CHF₂, t, J=233.3 Hz), 102.3 (C), 74.6 (CH), 70.5 (CH), 70.1 (CH), 68.7 (CH), 63.4 (CH₂), 53.9 (CH₃), 50.2 (CH), 39.5 (CH₂), 32.6 (CH₂), 30.2 (CH₂), 23.0 (CH₃), 21.3 (CH₃), 21.0 (CH₃), 21.0 (CH₃), 21.0 (CH₃); ¹⁹F NMR (CD₃OD): δ −115.4 (dd, J=320, 60 Hz), −117.6 (dd, J=320, 60 Hz); MS m/z (%): 733 (35, M⁺+H), 673 (24), 474 (23), 414 (100); HRMS calcd for C₃₁H₃₉F₂N₂O₁₆: 733.2268, found 733.2272.

A trifluoroacetic acid (TFA) salt of N-(6-aminohexyl)-5-(2-oxohexahydro-1H-thieno[3,4-d]imidazol-4-yl)pentanamide was prepared according to the procedures describe in Sabatino et al., J. Med. Chem. 2003, 46, 3170. To a solution of this TFA salt (124 mg, 0.27 mmol) and Intermediate VI (150 mg, 0.22 mmol) in anhydrous DMF (6.0 mL) was sequentially added 1-hydroxbenzotriazole (HOBt, 12 mg, 0.09 mmol), 1-ethyl-3-(3′-dimethylaminopropyl)carbodiimide (EDCI, 104 mg, 0.54 mmol), and diisopropylethylamine (DIEA, 0.15 mL, 0.85 mmol). After the reaction mixture was stirred at room temperature for 16 hours, the solvent was removed under reduced pressure. The resulting residue was purified by flash silica gel chromatography (10-30% gradient MeOH in CH₂Cl₂) to afford Intermediate VII: 3-acetoxy-5-acetylamino-2-[2-difluoromethyl-4-(3-{6-[5-(2-oxo-hexahydro-thieno[3,4-d]imidazol-6-yl)-pentanoylamino]-hexylcarbamoyl}-propionylamino)-phenoxy]-6-(1,2,3-triacetoxy-propyl)-tetrahydro-pyran-2-carboxylic acid methyl ester (177 mg, 75%) as a colorless foam. ¹H-NMR (CD₃OD, 400 MHz): δ 7.87 (d, J=2.3 Hz, 1 H, aromatic), 7.55 (dd, J=9.0, 2.3 Hz, 1 H, aromatic), 7.26 (d, J=9.0 Hz, 1 H, aromatic), 6.96 (t, J=55.3 Hz, 1 H, CHF₂), 5.38-5.36 (m, 2 H, H-7+H-8), 4.89-4.85 (m, 1 H), 4.51-4.48 (m, 2 H), 4.30-4.28 (m, 2 H), 4.09 (dd, J=14.0, 4.0 Hz, 1 H), 4.03 (dd, J=10.5, 10.5 Hz, 1 H), 3.64 (s, 3 H, OCH₃), 3.21-3.13 (m, 6 H), 2.91 (dd, J=12.8, 5.0 Hz, 1 H), 2.81 (dd, J=13.0, 4.6 Hz, 1 H), 2.71-2.65 (m, 3 H), 2.56-2.52 (m, 2 H), 2.20-2.16 (m, 2 H), 2.12 (s, 3 H, OAc), 2.10 (s, 3 H, OAc), 2.01 (s, 3 H, OAc), 1.99 (s, 3 H, OAc), 1.86 (s, 3 H, NAc), 1.76-1.32 (m, 14 H); ¹³C-NMR (CD₃OD, 100 MHz): δ 176.2 (C), 174.8 (C), 173.8 (C), 173.3 (C), 172.7 (C), 172.0 (C), 171.9 (C), 171.7 (C), 169.2 (C), 166.4 (C), 149.2 (C), 136.9 (C), 127.6 (C), 124.5 (CH), 121.9 (CH), 118.8 (CH), 112.9 (CHF₂, t, J=234.0 Hz), 102.3 (C), 74.6 (CH), 70.5 (CH), 70.1 (CH), 68.7 (CH), 63.4 (CH₂), 61.9 (CH), 57.3 (CH), 53.9 (CH₃), 50.3 (CH), 41.4 (CH₂), 40.6 (CH₂), 40.5 (CH₂), 39.5 (CH₂), 37.1 (CH₂), 33.3 (CH₂), 32.2 (CH₂), 30.6 (CH₂), 30.5 (CH₂), 30.1 (CH₂), 29.8 (CH₂), 27.9 (CH₂), 27.8 (CH₂), 27.2 (CH₂), 23.0 (CH₃), 21.4 (CH₃), 21.3 (CH₃), 21.1 (CH₃), 21.0 (CH₃); ¹⁹F NMR (CD₃OD): δ −115.3 (dd, J=324, 60 Hz), −117.5 (dd, J=324, 60 Hz); MS m/z (%): 1057 (20, M⁺+H), 663 (100), 647 (56); HRMS calcd for C₄₇H₆₇F₂N₆O₁₇S: 1057.4251 found 1057.4301.

Anhydrous Na₂CO₃ (26 mg, 0.24 mmol) was added to a solution of Intermediate VII (81 mg, 0.077 mmol) in dried MeOH (5.0 mL). After the mixture was stirred at room temperature for 2 hours, it was concentrated under reduced pressure to remove any volatile material. The residual mixture was dissolved in water (5.0 mL) and stirred for 16 hours. After the water was removed under reduced pressure, the resulting residue was purified by chromatography over Sephadex LH-20 using MeOH as an eluent to afford compound 1 (36 mg, 52%) as a white foam. ¹H-NMR (CD₃OD, 400 MHz) δ 7.79 (s, 1 H, aromatic), 7.51-7.45 (m, 2 H, aromatic), 7.10 (t, J=55.6 Hz, 1 H, CHF₂), 4.47 (dd, J=7.8, 4.6 Hz, 1 H), 4.28 (dd, J=7.8, 4.6 Hz, 1 H), 3.86-3.74 (m, 5 H), 3.63 (dd, J=11.5, 5.4 Hz, 1 H), 3.54 (d, J=9.2 Hz, 1 H), 3.20-3.12 (m, 5 H), 2.98 (dd, J=10.7, 3.2 Hz, 1 H), 2.90 (dd, J=11.8, 4.9 Hz, 1 H), 2.70-2.63 (m, 3 H), 2.52 (dd, J=7.2, 6.7 Hz, 2 H), 2.18 (t, J=7.3 Hz, 2 H), 2.00 (s, 3 H, NAc), 1.83-1.28 (m, 15 H); ¹³C-NMR (CD₃OD, 100 MHz): δ 176.3 (C), 175.9 (C), 174.8 (C), 173.1 (C), 166.4 (C), 150.5 (C), 136.2 (C), 128.7 (C, t, J=22 Hz), 124.1 (CH), 123.9 (CH), 118.2 (CH), 113.4 (CHF₂, t, J=233 Hz), 75.6 (CH), 73.4 (CH), 70.4 (CH), 69.5 (CH), 64.7 (CH₂), 63.6 (CH), 61.9 (CH), 57.3 (CH₃), 54.3 (CH), 42.9 (CH₂), 41.4 (CH₂), 40.6 (CH₂), 40.5 (CH₂), 37.1 (CH₂), 33.4 (CH₂), 32.4 (CH₂), 30.6 (CH₂), 30.5 (CH₂), 30.1 (CH₂), 29.8 (CH₂), 27.9 (CH₂), 27.8 (CH₂), 27.2 (CH₂), 22.9 (CH₃); ¹⁹F NMR (CD₃OD): δ −112.8 (dd, J=320, 60 Hz), −120.4 (dd, J=320, 60 Hz); MS m/z (%): 919 (23, M⁺+Na), 897 (42, M⁺+H), 606 (100); HRMS calcd for C₃₈H₅₆F₂N₆NaO₁₃S: 897.3492, found 897.3475.

### EXAMPLE 2

### Western Blot Analyses

Compound 1 was tested on its ability to bind neuraminidase obtained from Athrobacter ureafaciens. Athrobacter ureafaciens neuraminidase (0.8 U, Sigma, St. Louis, Mo.) was incubated in the presence or absence of compound 1 (200 μM) at 4° C. in 10 mL of an ammonium acetate buffer (100 mM). Bovine serum albumin (BSA, 0.65 μg/μl) was used as a negative control, and was also prepared in the presence or absence of compound 1 (200 μM) at 4° C. in 10 mL of an ammonium acetate buffer (100 mM). Four samples were tested using the Western blot analysis: (1) Athrobacter ureafaciens neuraminidase alone, (2) BSA alone, (3) Athrobacter ureafaciens neuraminidase and compound 1, and (4) BSA and compound 1. Each sample was applied to 10% polyacrylamide gel followed by SDS-PAGE. After electrophoresis, the protein was transferred from the gel onto a PVDF membrane. The PVDF membrane was blocked, washed, and developed using ECL Western blot protocols (Amersham Biosciences, Pittsburgh, Pa.) as recommended by the supplier.

The results show that sample (3) exhibited three bands on the PVDF membrane, which correspond to three biotinylated isoenzymes of Athrobacter ureafaciens neuraminidase having molecular weights of 88, 66, and 52 KDa, respectively. The results also show that no band was observed for samples (1) and (2) (which contained no compound 1) and sample (4) (which contained BSA and compound 1).

### EXAMPLE 3

### Inhibition Assays

3.3 mM of compound 1 or zanamivir (Glaxo Wellcome Research and Development Ltd, Stevenage, United Kingdom) was pre-incubated for 45 minutes with influenza A virus (A/WSN/33; 9×10³ PFU), Athrobacter ureafaciens neuraminidase (5 mU), Clostridium perfringens neuraminidase (10 U), Vibro cholerae neuraminidase (3.7 mU) in MES buffer (32.5 mM MES, pH 6.5, 4 mM CaCl₂), respectively. The reaction was initiated by addition of a small aliquot of 4-methylumbelliferyl-N-acetylneuraminic acid (3.3 μM MUNANA, Sigma Chemical Co., St. Louis, Mo.) to a 150 μL solution prepared above in a black 96-well plate. After 2-hour of incubation at 37° C., the reaction was stopped by the addition of 100 μL of freshly prepared 0.14 M NaOH in 83% ethanol. Fluorometric measurement was carried out immediately using a fluorometer (Fluoroskan Ascent from ThermoLabsystems, Helsinki, Sweden). The excitation wavelength and the emission wavelength used during the measurement were 355 nm and 460 nm, respectively. Unexpectedly, the results showed that compound 1 exhibited significant inhibitory effect on the activities of influenza A virus neuraminidase, as well as the other three neuraminidases. By contrast, zanamivir exhibited strong inhibitory effect on the activity of influenza A virus neuraminidase, but only weak inhibitory effect on the activities of the other three neuraminidases. The results indicate that compound 1 retains its inhibitory activity to different neuraminidases, while zanamivir's binding interaction with influenza A virus neuraminidase is greatly reduced against other neuraminidases.

Experiments for determining the IC₅₀ value (i.e., the fifty percent inhibitory concentration) of compound 1 against the above-mentioned four neuraminidases were carried out in a manner similar to that describe above except that a different buffer was used. Sepcifically, compound 1 (0-3.3 mM) was respectively incubated with influenza A virus in 32.5 mM MES buffer, pH 6.5, and with Athrobacter ureafaciens, Clostridium perfringens, and Vibro cholerae in 80 mM sodium acetate buffer, pH 5.0. The residual activities of the neuraminidases were measured as described above and the IC₅₀ values were calculated from concentration-response curves using Microcal Origin Software. The results show that compound 1 had IC₅₀ values of 1.7, 0.68, 0.08, and 0.53 mM against neuraminidases of influenza A virus, Athrobacter ureafaciens, Clostridium perfringens, and Vibro cholerae, respectively.

Experiment for determining the IC₅₀ value of zanamivir against influenza A virus neuraminidase was carried out in the same manner as that describe above. The results indicate that zanamivir had a IC₅₀ value of about 3.1 nM against influenza A virus neuraminidase.

### EXAMPLE 4

### ELISA Assay

Compound 1 (5 nmol) was added to wells of a streptavidin coated 96-well ELISA plate (NUNC IMMOBILIZER, Rochester, N.Y.). BSA-biotin conjugate was used as a negative control. After an 1-hour incubation, the wells were blocked with 0.1% BSA/phosphate buffered saline (PBS) for 1 hour and wash with PBS. An influenza A virus (A/WSN/33, 3.8×10²-970×10² PFU) solution was added to the wells. The mixture was then incubated for 1 hour at room temperature. After another wash with PBS, captured viruses were detected by sequential treatments with a polyclonal anti-Flu A antibody, a goat antirabbit-horseradish peroxidase conjugate, and a TMB substrate. The results indicated that compound 1 bound to the plate wells successfully captured influenza A virus. The intensity of responding signals was proportional to the amount of influenza A virus added to the wells. By contrast, the wells loaded with BSA-biotin conjugate gave negative response.

A selective capturing experiment using a mixture of influenza A virus and Japanese encephalitis virus (JEV) was conducted in a manner similar to that described above. Unlike influenza A virus, JEV does not contain neuraminidase on its surface. Anti-Flu A and anti-JEV antibodies were used to detect any captured influenza A virus and JEV, respectively. The results show that only influenza A virus, but not JEV, was captured and detected on the plate, indicating that capture of virus particles resulted from the interaction between compound 1 and the neuraminidase on the surface of the virus.

Another capturing experiment was conducted using influenza A virus and influenza A virus whose active site on neurminidase was blocked by zanamivir. Specifically, influenza viruse (9×10³ PFU) was pre-incubated in the presence or absence of zanamivir (67 μM) for 45 minutes and than treated with compound 1 (667 μM) for another 45 minutes. After incubation, the mixture was added to a NUNC streptavidin plate and incubated for 1 hour. After the mixture was washed three times with PBS, anti-biotin HRP (1:1000 dilute) was added to the mixture. Captured viruses were detected by adding TMB substrate (50 μM) and measuring the optical density. The results show that compound 1 can capture influenza A virus 14 folds as much as influenza A virus pretreated with zanamivir, indicating that compound 1 attached to influenza A virus by binding to the active site of its neuraminidase.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 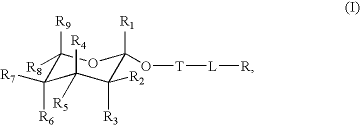
wherein
T is arylene or C₇-C₂₀ arylalkylene;
L is -L₁-L₂-L₃-; L₁ being deleted, —C(O)N(Rₐ₁)—, or —N(Rₐ₁)C(O)—; L₂ being deleted or C₁-C₃₀ alkyl optionally containing 1-10 heteroatoms, —C(O)N(Rₐ₂)—, or —N(Rₐ₂)C(O)—; and L₃ being deleted or —N(Rₐ₃)—;
R is —C(O)—R_{b1}, —S(O)₂—R_{b1}, —N(R_{b1})(R_{b2}), —N₃, C₂-C₁₀ alkynyl, or heteroaryl;
R₁ is COOR_{c1};
each of R₂ and R₃, independently, is H, OR_{d1}, or C₁-C₁₀ alkyl;
one of R₄ and R₅ is ORₑ₁, and the other of R₄ and R₅ is H, ORₑ₂, or C₁-C₁₀ alkyl;
one of R₆ and R₇ is N(R_{f1}R_{f2}), and the other of R₆ and R₇ is H, OR_{f3}, or C₁-C₁₀ alkyl; and
one of R₈ and R₉ is C₁-C₁₀ alkyl substituted with OR_{g1}, and the other of R₈ and R₉ is H, OR_{g2}, or C₁-C₁₀ alkyl;
in which each of Rₐ₁, Rₐ₂, Rₐ₃, R_{b1}, R_{b2}, R_{c1}, R_{d1}, Rₑ₁, Rₑ₂, R_{f1}, R_{f2}, R_{f3}, R_{g1}, and R_{g2}, independently, is H, C₁-C₁₀ alkyl, C₃-C₂₀ cycloalkyl, C₃-C₂₀ heterocycloalkyl, heteroaryl, aryl, or —C(O)R′; R′ being H or C₁-C₁₀ alkyl;
or a salt thereof.

2. The compound of claim 1, wherein T is arylene.

3. The compound of claim 2, wherein T is phenylene substituted with CHF₂.

4. The compound of claim 3, wherein L₁ is —N(Rₐ₁)C(O)—, L₂ is C₁-C₃₀ alkyl containing —C(O)N(Rₐ₂)— and —N(Rₐ₂)C(O)—, and L₃ is —N(Ra₃)—.

5. The compound of claim 4, wherein R is —C(O)—R_{b1}.

6. The compound of claim 5, wherein R_{b1} is C₁-C₁₀ alkyl substituted with heteroaryl.

7. The compound of claim 6, wherein R₁ is COOH, R₂ is H, R₃ is H, R₄ is H, R₅ is OH, R₆ is H, R₇ is NHAc, R₈ is C₁-C₁₀ alkyl substituted with three OH, and R₉ is H.

8. The compound of claim 7, wherein the compound is 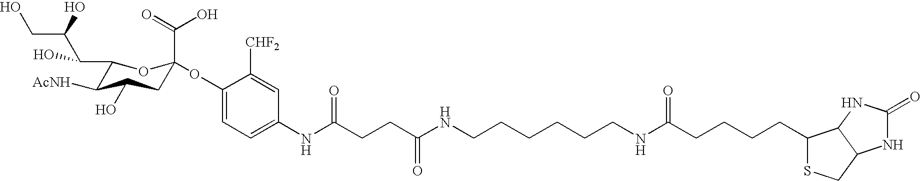
or a salt thereof.

9. The compound of claim 1, wherein T is C₇-C₂₀ arylalkylene.

10. The compound of claim 9, wherein T is

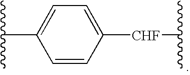

11. The compound of claim 10, wherein L₁ is —C(O)N(Rₐ₁)—, L₂ is C₁-C₃₀ alkyl containing 1-10 heteroatoms, and L₃ is —N(Rₐ₃)—.

12. The compound of claim 11, wherein R is —C(O)—R_{b1}.

13. The compound of claim 12, wherein R_{b1} is C₁-C₁₀ alkyl substituted with heteroaryl.

14. A method of treating an infection with an influenza virus, comprising administering to a subject in need thereof an effective amount of a compound of formula (I): 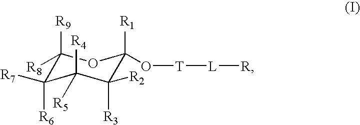
wherein
T is arylene or C₇-C₂₀ arylalkylene;
L is -L₁-L₂-L₃-; L₁ being deleted, —C(O)N(Rₐ₁)—, or —N(Rₐ₁)C(O)—; L₂ being deleted or C₁-C₃₀ alkyl optionally containing 1-10 heteroatoms, —C(O)N(Rₐ₂)—, or —N(Rₐ₂)C(O)—; and L₃ being deleted or —N(Rₐ₃)—;
R is —C(O)—R_{b1}, —S(O)₂—R_{b1}, —N(R_{b1})(R_{b2}), —N₃, C₂-C₁₀ alkynyl, or heteroaryl;
R₁ is COOR_{c1};
each of R₂ and R₃, independently, is H, OR_{d1}, or C₁-C₁₀ alkyl;
one of R₄ and R₅ is ORₑ₁, and the other of R₄ and R₅ is H, ORₑ₂, or C₁-C₁₀ alkyl;
one of R₆ and R₇ is N(R_{f1}R_{f1}), and the other of R₆ and R₇ is H, OR_{f3}, or C₁-C₁₀ alkyl; and
one of R₈ and R₉ is C₁-C₁₀ alkyl substituted with OR_{g1}, and the other of R₈ and R₉ is H, OR_{g2}, or C₁-C₁₀ alkyl;
in which each of Rₐ₁, Rₐ₂, Rₐ₃, R_{b1}, R_{b2}, R_{c1}, R_{d1}, Rₑ₁, Rₑ₂, R_{f1}, R_{f2}, R_{f3}, R_{g1}, and R_{g2}, independently, is H, C₁-C₁₀ alkyl, C₃-C₂₀ cycloalkyl, C₃-C₂₀ heterocycloalkyl, heteroaryl, aryl, or —C(O)R′; R′ being H or C₁-C₁₀ alkyl;
or a salt thereof.

15. The method of claim 14, wherein T is arylene.

16. The method of claim 15, wherein T is phenylene substituted with CHF₂.

17. The method of claim 16, wherein L₁ is —N(Rₐ₁)C(O)—, L₂ is C₁-C₃₀ alkyl containing —C(O)N(Rₐ₂)— and —N(Rₐ₂)C(O)—, and L₃ is —N(Rₐ₃)—.

18. The method of claim 17, wherein R is —C(O)—R_{b1}.

19. The method of claim 18, wherein R_{b1} is C₁-C₁₀ alkyl substituted with heteroaryl.

20. The method of claim 19, wherein R₁ is COOH, R₂ is H, R₃ is H, R₄ is H, R₅ is OH, R₆ is H, R₇ is NHAc, R₈ is C₁-C₁₀ alkyl substituted with three OH, and R₉ is H.

21. The method of claim 14, wherein T is C₇-C₂₀ arylalkylene.

22. The method of claim 21, wherein T is

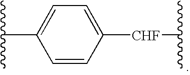

23. The method of claim 22, wherein L₁ is —C(O)N(Rₐ₁)—, L₂ is C₁-C₃₀ alkyl containing 1-10 heteroatoms, and L₃ is —N(Rₐ₃)—.

24. The method of claim 23, wherein R is —C(O)—R_{b1}.

25. The method of claim 24, wherein R_{b1} is C₁-C₁₀ alkyl substituted with heteroaryl.

26. A method of detecting presence of an influenza virus in a sample, comprising:
contacting a sample with a compound of formula (I): 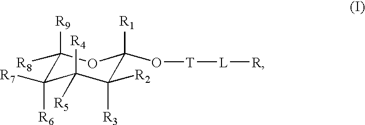
wherein
T is arylene or C₇-C₂₀ arylalkylene;
L is -L₁-L₂-L₃-; L₁ being deleted, —C(O)N(Rₐ₁)—, or —N(Rₐ₁)C(O)—; L₂ being deleted or C₁-C₃₀ alkyl optionally containing 1-10 heteroatoms, —C(O)N(Rₐ₂)—, or —N(Rₐ₂)C(O)—; and L₃ being deleted or —N(Rₐ₃)—;
R is —C(O)—R_{b1}, —S(O)₂—R_{b1}, —N(R_{b1})(R_{b2}), —N₃, C₂-C₁₀ alkynyl, or heteroaryl;
R₁ is COOR_{c1};
each of R₂ and R₃, independently, is H, OR_{d1}, or C₁-C₁₀ alkyl;
one of R₄ and R₅ is ORₑ₁, and the other of R₄ and R₅ is H, ORₑ₂, or C₁-C₁₀ alkyl;
one of R₆ and R₇ is N(R_{f1}R_{f2}), and the other of R₆ and R₇ is H, OR_{f3}, or C₁-C₁₀ alkyl; and
one of R₈ and R₉ is C₁-C₁₀ alkyl substituted with OR_{g1}, and the other of R₈ and R₉ is H, OR_{g2}, or C₁-C₁₀ alkyl;
in which each of Rₐ₁, Rₐ₂, Rₐ₃, R_{b1}, R_{b2}, R_{c1}, R_{d1}, Rₑ₁, Rₑ₂, R_{f1}, R_{f2}, R_{f3}, R_{g1}, and R_{g2}, independently, is H, C₁-C₁₀ alkyl, C₃-C₂₀ cycloalkyl, C₃-C₂₀ heterocycloalkyl, heteroaryl, aryl, or —C(O)R′; R′ being H or C₁-C₁₀ alkyl; or a salt thereof; and
determining presence of binding between the compound of formula (I) and an influenza virus, the binding being an indication of presence of the influenza virus.

27. The method of claim 26, wherein T is arylene.

28. The method of claim 27, wherein T is phenylene substituted with CHF₂.

29. The method of claim 28, wherein L₁ is —N(Rₐ₁)C(O)—, L₂ is C₁-C₃₀ alkyl containing —C(O)N(Rₐ₂)— and —N(Rₐ₂)C(O)—, and L₃ is —N(Rₐ₃)—.

30. The method of claim 29, wherein R is —C(O)—R_{b1}.

31. The method of claim 30, wherein R_{b1} is C₁-C₁₀ alkyl substituted with heteroaryl.

32. The method of claim 31, wherein R₁ is COOH, R₂ is H, R₃ is H, R₄ is H, R₅ is OH, R₆ is H, R₇ is NHAc, R₈ is C₁-C₁₀ alkyl substituted with three OH, and R₉ is H.

33. The method of claim 26, wherein T is C₇-C₂₀ arylalkylene.

34. The method of claim 33, wherein T is

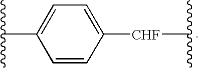

35. The method of claim 34, wherein L₁ is —C(O)N(Rₐ₁)—, L₂ is C₁-C₃₀ alkyl containing 1-10 heteroatoms, and L₃ is —N(Rₐ₃)—.

36. The method of claim 35, wherein R is —C(O)—R_{b1}.

37. The method of claim 36, wherein R_{b1} is C₁-C₁₀ alkyl substituted with heteroaryl.

38. The method of claim 26, wherein the method includes a western blot analysis.

39. The method of claim 26, further comprising attaching the compound of formula (I) to a substrate before the contacting step.

40. The method of claim 39, wherein the method includes an enzyme-linked immunosorbent assay.
